(19) United States
(12) Patent Application Publication
(10) Pub. No.: US 2025/0303046 A1
(43) Pub. Date: Oct. 2, 2025

(54) COMPUTER-IMPLEMENTED METHOD FOR A DIALYSIS SYSTEM

(71) Applicant: B. Braun Avitum AG, Melsungen (DE)

(72) Inventors: Waldemar Janik, Melsungen (DE); Joshua Lindner, Kassel (DE)

(21) Appl. No.: 19/090,706

(22) Filed: Mar. 26, 2025

(30) Foreign Application Priority Data
Mar. 28, 2024 (DE) 10 2024 109 052.9

(51) Int. Cl. A61M 1/36; A61M 1/14

(52) U.S. Cl. CPC A61M 1/3627; A61M 1/14; A61M 2205/3337; A61M 2205/3368; A61M 2205/36; A61M 2205/50

(57) ABSTRACT

A computer-implemented method for a dialysis system is used for generating control data for pressure-based control of the dialysis system. The method includes the steps of monitoring pressure in a degassing section of a fluid system of the dialysis system, and generating control data when the pressure is detected to meet at least one criterion. The step of generating the control data can include generating a heating device control signal for a heating device of the dialysis system.

### CROSS-REFERENCE TO RELATED APPLICATION

This application claims priority under 35 U.S.C. § 119 to German Application No. 10 2024 109 052.9, filed on Mar. 28, 2024, the content of which is incorporated by reference herein in its entirety.

### FIELD

The present disclosure relates to a computer-implemented method for a dialysis system, a system for data processing, a dialysis system comprising the system for data processing, a computer program product and a computer-readable medium.

### BACKGROUND

Controlling the various components in dialysis systems is always a challenge. The control of heating devices and valves is particularly important.

For example, current systems rely on monitoring the fill level in a chamber to control the heating process. A mechanical level sensor with a float is often used. For example, the control can be carried out in such a way that the heating device is only switched on when the level sensor has detected that a corresponding fill level has been reached in the chamber. As a further example, the control can be carried out in such a way that the heating device is switched off as soon as a level falls below the fill level. The control of valves, especially an inlet valve, is also relevant in addition to the heating control and is often linked to the measured values of the level sensor because a valve status also determines the inflow and/or outflow of the fluid and therefore the fill level.

However, the use of mechanical level sensors has the disadvantage that they are not always reliable, in particular they are prone to errors such as the float jamming, which would register an incorrect fill level. There is therefore a need for more reliable control of the heating device and, where appropriate, valves.

Operating the heating device with too little fluid can cause damage to the system and/or lead to malfunctions, some of which are also safety-relevant. There is a need to prevent such damage and malfunctions more reliably.

The present disclosure is based on the task of providing a computer-implemented method for a dialysis system which addresses at least some of the problems listed above, in particular enables reliable control and/or reliably prevents damage and/or malfunctions

### SUMMARY

The present disclosure provides a computer-implemented method for a dialysis system, a system for data processing, a dialysis system comprising the system for data processing, a computer program product and a computer-readable medium.

The present disclosure relates to a computer-implemented method for a dialysis system for generating control data for pressure-based control of the dialysis system, the method comprising: monitoring pressure in a degassing section of a fluid system of the dialysis system, generating control data when it is detected that the pressure meets at least one criterion, wherein generating the control data comprises generating a heating device control signal for a heating device of the dialysis system. In the degassing section, fluid, for example water, can be tempered.

In other words, a method is provided which generates control data by means of which the heating device of the dialysis system can be controlled as a function of pressure, in particular, for example, started and/or stopped as a function of pressure. The control of the heating device is achieved by means of the heating device control signal. The heating device control signal is generated depending on the pressure. The control data, in particular the heating device control signal, is generated when it is detected that the pressure being monitored in the degassing section meets a criterion, for example drops and/or rises too quickly and/or too far.

The pressure in the degassing section is an indicator of whether at least a predetermined minimum amount of fluid is present in the fluid system, in particular in an upstream container from which fluid enters the degassing section. It is therefore possible to indirectly deduce the fill level from pressure measurement values, which enables more reliable control, as the pressure measurements can be used as an alternative or in addition to the potentially unreliable level sensor.

The dialysis system can be configured in such a way that, in normal operation, the fluid flows through the degassing section before it enters the heating device for heating. Therefore, if there is a change in pressure in the degassing section that indicates that there is too little fluid in the fluid system, this also indicates in particular that there is also an increased probability that there is too little fluid in a heating container in which the fluid is heated by the heating device. For the reasons explained above, this can be detected more reliably than before. This can also reduce the risk of the heating device heating with too little fluid and thus reduce the risk of damage and malfunctions.

Coupling the operation of the heating device (and optionally the valves) to the pressure in the degassing section therefore makes it possible to address at least the problems mentioned above, in particular to enable reliable control and/or reliably prevent damage and/or malfunctions.

It should be noted here that in the present disclosure, unless explicitly stated otherwise, the features of the method are directed in particular to the intended operation of the dialysis system configured as intended.

The dialysis system can be configured at least for collecting, degassing and heating fluid as part of an extracorporeal blood treatment. The dialysis system can also be configured to add concentrates for the production of dialysis fluid and/or for balancing including ultrafiltration. The dialysis system can comprise a blood pump, a dialyzer, a pressure sensor and/or a tube system. The dialysis system may also comprise one or more actuators and/or one or more (further) sensors for carrying out a dialysis treatment.

According to the present disclosure, a generation of control data for controlling the dialysis system may comprise at least the generation of a control signal for a component of the dialysis system, for example the heating device control signal. The control data may comprise one or more control signals for one or more components of the dialysis system.

In the following, the term “control signal” is used collectively for the respective control signals for different components (e.g. heating device control signal and valve control signal).

In the present disclosure, it is assumed in particular that the control signal is used directly, i.e. without time delay, to control the component by means of the control signal.

Fulfilling the criterion can trigger immediate generation of the control data, in particular the control signal. This is also referred to as triggering without delay. Optionally, the immediately generated control data, in particular the control signal, may comprise a predetermined delay after which the component is to perform an action predetermined by the control signal, for example start component operation, stop component operation, open (if the component is a valve), and/or close (if the component is a valve). For the control signals for different components, any delay specified by the respective control signal may be different.

The pressure sensor or pressure sensors can be arranged in one or more areas of the degassing section.

The degassing section can comprise a degassing chamber, a pump and a throttle. The pressure can, for example, be measured in the direction of flow between the throttle and the pump. The degassing chamber can be arranged in the direction of flow between the pressure measurement area and the pump.

A fluid container can be positioned upstream of the degassing section in the direction of flow, from which fluid is supplied to the degassing section during operation. When the fluid container is sufficiently full and the pump is running, a pressure value is reached in the degassing section that is lower than the pressure value when the fluid container is empty. A change in pressure therefore indicates a change in the fill level in the container.

Monitoring the pressure can include, for example, regular or continuous recording of measured values from one or more pressure sensors. Monitoring the pressure may involve at least one pressure sensor being arranged in the degassing section and measuring the pressure at, for example, regular intervals or continuously and providing measured values. The degassing chamber may be a degassing chamber commonly used in this field.

The method may include using the values obtained by monitoring the pressure to check whether the pressure fulfills the (at least one) criterion. The criterion may relate to the obtained values themselves or to data derived from the obtained values, for example by averaging, determining the slope, determining the standard deviation, integration, or the like. Derived data may, for example, include data representing a time course of the values obtained.

The (at least one) criterion can be retrieved from a memory for testing. The criterion can be a theoretically determined criterion, for example by modeling the dialysis system, or an empirically determined criterion or a semi-empirically determined criterion. For example, the criterion can be derived from the dimensions of the system and the properties of the installed components. Alternatively, the criterion can be selected independently of the dimensions and properties, i.e. in such a way that it is suitable for all expected dimensions and properties

Examples are explained below.

In particular, the heating device of the dialysis system may comprise an electrically operated heating device. The heating device can be configured and arranged in such a way that it heats fluid, in particular fluid that is in the heating container or flows through the heating container, during intended operation. For example, the heating device can comprise a heating rod that protrudes into the heating container or a heating element, for example a heating tube, which can be formed integrally with the container wall or arranged on the container wall. Alternative embodiments are also conceivable.

According to the present disclosure, the at least one criterion may comprise a first criterion relating to a pressure value.

Such a criterion can also be regarded as a static criterion, which refers to a state of the pressure at a point in time or in a time interval of a specified length. The use of a static criterion enables a simple, efficient and reliable check as to whether the criterion is fulfilled. For example, the criterion can be that the pressure value in a time interval corresponds to a target pressure value or is within a target interval or is above or below a target value. This is described in detail below.

Alternatively or additionally, the at least one criterion may comprise a second criterion relating to a pressure change, in particular a pressure increase and/or a pressure drop.

Such a criterion can also be considered as a dynamic criterion, which refers to the change, for example decrease or increase, of the pressure value, especially over a time interval of a predetermined length. A dynamic criterion enables a test that is less susceptible to individual measurement errors. Depending on the criterion, it is even possible to obtain correct results even if, for example, the absolute measured pressure is not correct, as the change is independent of the absolute value.

According to the present disclosure, a pressure value can in particular be a measured value of one or more pressure sensors or a pressure value determined from one or more measured values of one or more pressure sensors. For example, the pressure value can be a pressure value determined statistically from several measured values of a pressure sensor, for example an average value over a predetermined time interval.

According to the present disclosure, the at least one criterion, in particular the first criterion or the static criterion, may comprise a criterion that an actual pressure value corresponds to a target pressure value, optionally over a time interval with a predetermined length.

Alternatively or additionally, the at least one criterion, in particular the first criterion or static criterion, can comprise a criterion that the pressure value corresponds to a target pressure value, is within a target interval, and/or is greater than the target pressure value or less than the target pressure value, optionally in each case over a time interval of a predetermined length.

Alternatively or additionally, according to the present disclosure, the at least one criterion, in particular the second criterion or dynamic criterion, may comprise a criterion that an actual pressure value falls below a predetermined pressure value or that an actual pressure value rises above a predetermined pressure value or that an actual pressure value changes by at least a predetermined amount, in particular rises or falls by at least the predetermined amount, in particular within a time interval of predetermined length. Alternatively or additionally, the at least one criterion, in particular the second criterion or dynamic criterion, may comprise a criterion that the pressure value changes faster than a predetermined rate of change, in particular within an interval of predetermined length and/or at least by a predetermined minimum amount.

Alternatively or additionally, according to the present disclosure, the at least one criterion, in particular the second criterion or dynamic criterion, may comprise a criterion that a moving standard deviation of the pressure rises above a predetermined value. This standard deviation refers to several pressure values measured consecutively over a period of time.

If a criterion is used that is focused, for example, on the amount of change and/or the rate of change of the change in the pressure value, this makes it possible to obtain reliable results independently of an absolute pressure measurement.

If a criterion refers to the moving standard deviation, this makes it possible not to use outliers or fluctuations in measured values as triggers for generating the control signal, but to reliably detect real pressure changes, for example, and only generate control data based on such real pressure changes. The use of the standard deviation is particularly advantageous because the intake of air bubbles can cause the actual value to fluctuate significantly.

According to the present disclosure, the at least one criterion may comprise a start criterion for starting the heating device, wherein when the pressure satisfies a start criterion, a start signal is generated as a heating device control signal.

This means that if it is detected that the start criterion is fulfilled, the starting of the heating device can be triggered immediately or with a predefined delay (for example according to the control signal, here according to the start signal). Starting the heating device is to be regarded here in particular as starting a heating process of the heating device.

As explained above for the control signals in general, the start signal can be generated immediately. The start signal can be configured in such a way that it causes the heating device to start immediately or after a delay specified by the start signal, in particular to start the heating process.

In particular, the start criterion can be selected such that it relates to a pressure value and/or a pressure change, for example comprising the first criterion explained above and/or the second criterion explained above. In particular, the start criterion can be selected such that the pressure value and/or the pressure change, for example in general or for the present system, indicates that fluid is present in the container upstream of the degassing section in the flow direction.

Alternatively or in addition to a start criterion, according to the present disclosure, the at least one criterion may comprise a stop criterion for stopping the heating device, wherein when the pressure satisfies a stop criterion, a stop signal is generated as a heating device control signal.

This means that if it is recognized that the stop criterion is fulfilled, the stopping of the heating device can be triggered immediately or with a predefined delay (for example according to the control signal, here according to the stop signal). Stopping the heating device is to be regarded here in particular as stopping a heating process of the heating device.

As explained above for the control signals in general, the stop signal can be generated immediately. The stop signal can be configured in such a way that it causes the heating device to stop immediately or after a delay specified by the stop signal, in particular to stop the heating process.

The stop criterion can be selected in particular in such a way that it relates to a pressure value and/or a pressure change, for example comprising the first criterion explained above and/or the second criterion explained above. In particular, the stop criterion can be selected such that the pressure value and/or the pressure change, for example in general or for the present system, indicates that there is no or too little fluid in the container upstream of the degassing section in the flow direction.

Alternatively or in addition to a start criterion and/or stop criterion, according to the present disclosure, the at least one criterion may comprise an adjustment criterion for adjusting the operation of the heating device, wherein when the pressure satisfies an adjustment criterion, an adjustment signal is generated as a heating device control signal.

This means that if it is detected that the adaptation criterion has been met, the operation of the heating device can be adapted immediately or with a predetermined delay (for example according to the control signal, in this case according to the adaptation signal). The adjustment may involve changing one or more operating parameters of the heating device in operation. For example, the adjustment may comprise changing a heating power and/or a setpoint temperature as operating parameters of the heating device.

As explained above for the control signals in general, the adjustment signal can be generated immediately. The adjustment signal can be configured in such a way that it causes the heating device to carry out the adjustment immediately or after a delay specified by the adjustment signal, in particular to change the operating parameter(s).

In particular, the adjustment signal may be selected such that it relates to a pressure value and/or a pressure change, for example comprising the first criterion explained above and/or the second criterion explained above. In particular, the adaptation signal can be selected such that the pressure value and/or the pressure change, for example in general or for the present system, indicates that the amount of fluid in the container upstream of the degassing section in the flow direction has changed, for example changed by more than a threshold value.

According to the present disclosure, the at least one criterion may comprise the start criterion and the start criterion may comprise that an actual pressure value falls below a predetermined pressure value or that an actual pressure value falls by at least a predetermined amount or that a moving standard deviation of the pressure rises above a predetermined value. The temporal standard deviation can refer to several pressure values recorded consecutively over a period of time.

A change in pressure is particularly meaningful, as a change indicates that the previous state, i.e. the presence or lack of fluid, has changed or is changing significantly.

According to the present disclosure, generating the control data may comprise generating a valve control signal for an inlet valve of the dialysis system that controls the supply of fluid into a fluid system of the dialysis system.

In intended use, the opening of the inlet valve controls the inflow of fluid into the fluid system, in particular into the container(s) upstream of the degassing section in the flow direction, which is part of the fluid system. Thus, the valve control signal can be used to start or stop a fluid supply by controlling the inlet valve accordingly. As already explained above, the pressure, in particular the pressure value and/or a change in pressure, is an indicator of the presence or absence of fluid. Coupling the generation of the valve control signal to the pressure therefore makes it possible to supply fluid if the pressure indicates that there is an absence of fluid and/or to interrupt the supply of fluid if the pressure indicates that there is a sufficient amount of fluid. Corresponding criteria are explained below.

In case generating the control data comprises generating a valve control signal for an inlet valve of the dialysis system that controls the fluid supply into a fluid system of the dialysis system, the at least one criterion may comprise an open criterion, wherein when the pressure satisfies the open criterion, a signal to open the inlet valve is generated. Alternatively or additionally, the at least one criterion may comprise a closing criterion, wherein if the pressure fulfills the closing criterion, a signal to close the inlet valve is generated.

This means that if it is detected that the opening criterion is met, the opening of the inlet valve can be triggered immediately or with a predetermined delay, and/or if the closing criterion is met, the closing of the inlet valve can be triggered immediately or with a predetermined delay. As explained above for the control signals in general, the valve control signal can be generated immediately. The valve control signal can be configured in such a way that it causes the inlet valve to open or close immediately or after a delay specified by the valve control signal.

The opening criterion and/or the closing criterion can in each case be selected in particular in such a way that it relates to a pressure value or a pressure change, for example comprising the first criterion explained above or the second criterion explained above. In particular, the opening criterion can be selected such that the pressure value or the pressure change, for example in general or for the system in question, indicates that there is no or too little fluid in the container upstream of the degassing section in the flow direction. Alternatively or additionally, the closing criterion can be selected in such a way that the pressure value or the pressure change, for example in general or for the system in question, indicates that there is sufficient fluid in the container upstream of the degassing section in the flow direction.

The method of the present disclosure may comprise controlling the heating device by means of the heating device control signal and/or controlling the inlet valve by means of a/the valve control signal.

Reference is made inter alia to the above explanations. Controlling the heating device by means of the heating device control signal may involve starting, stopping or adjusting the operation of the heating device in accordance with the heating device control signal. This can be done immediately or with a delay specified by the heating device control signal. Controlling an/the inlet valve by means of a/the valve control signal may comprise opening or closing the inlet valve in accordance with the valve control signal. This can take place immediately or with a delay specified by the valve control signal

According to the present disclosure, controlling the heating device may comprise controlling the heating device according to the heating device control signal with a predetermined delay and/or after meeting at least one further criterion and/or after user confirmation or immediately upon meeting the at least one criterion, in particular starting or stopping or adjusting the heating according to the heating device control signal.

In this context, reference is also made to the explanations above. In particular, the delay can be achieved by a delay predetermined by the heating device control signal. In particular, if the heating device is controlled after at least one further criterion has been met, the heating device control signal can follow immediately after the further criterion has been met.

For example, it is conceivable that two pressure-related criteria or one pressure-related criterion and one non-pressure-related criterion must be fulfilled for the heating device control signal to be generated.

Controlling by user confirmation may include, for example, once it is detected that the criterion is met, issuing a request to a user to confirm generating the heating device control signal and/or controlling the heating device accordingly.

According to the present disclosure controlling the inlet valve may comprise controlling the inlet valve according to the valve control signal with a predetermined delay and/or after at least one further criterion has been met and/or after user confirmation or immediately upon meeting the at least one criterion, in particular opening or closing the valve according to the valve control signal.

In this context, reference is made to the explanations above. In particular, the delay can be achieved by a delay specified by the valve control signal. In particular, if the inlet valve is controlled after at least one further criterion has been met, the valve control signal can follow immediately after the further criterion has been met.

For example, it is conceivable that two pressure-related criteria or one pressure-related criterion and one non-pressure-related criterion must be fulfilled for the valve control signal to be generated.

Control after user confirmation can include, for example, that once it is recognized that the criterion is met, a request is issued to a user to confirm to generate the valve control signal and/or to control the inlet valve accordingly.

According to the present disclosure, the method may comprise a testing process for testing a level sensor in a fluid container of the dialysis system, in particular a container upstream of the degassing section in the flow direction, wherein the monitored pressure and/or the control data are used as first input data for the testing process, wherein measurement data of the level sensor are used as second input data for the testing process, wherein the testing process outputs that the level sensor is functioning properly,
if both the measurement data of the level sensor and the pressure and/or the control data indicate that the amount of fluid in the dialysis system has fallen below a specified minimum level, or
if both the measurement data of the level sensor and the pressure and/or the control data indicate the presence of a specified minimum amount of fluid in the dialysis system,
   otherwise the testing process will indicate that the level sensor is not functioning properly.

In other words, the testing process will make it possible to check that the different determination methods match. If there is no agreement, a non-functioning level sensor can be assumed. the pressure sensor as a reference is advantageous because a malfunction of the pressure sensor can be recognized more reliably than a malfunction of a level sensor.

According to the present disclosure, in particular always or at least in a case in which the/one level sensor and/or the/one temperature sensor fails, a control of the heating and/or of an/inlet valve can take place partially or completely according to the monitored pressure.

In normal operation, for example, the inlet valve can be controlled according to the measurement of a/the level sensor in the heating container and/or the heating device according to a temperature measurement of a/the temperature sensor. As the fluid level or temperature is directly linked to the fluid supply or the heating operation of the heating device, this is a simple variant. However, it is conceivable, at least in emergency operation, i.e. when control based on measured level values or temperatures is not possible, and optionally also in normal operation, to take the monitored pressure into account for the control, in particular to carry out the control without the use of measured level values or temperatures.

In this way, greater reliability can be achieved.

According to the present disclosure, only the starting and optionally also the stopping of the heating can be pressure-dependent according to the monitored pressure and the adjustment of the heating can be pressure-independent during heating operation. This applies in particular to normal operation. In normal operation, for example, temperature-based control, in particular adjustment of the heating device, can take place during heating operation, wherein the temperature can be measured in the heating container, for example.

The combination enables simple and immediate adjustment of the heating power to provide the desired temperature and the possibility of starting and, where appropriate, stopping heating independently of the temperature, which is not always a reliable criterion for these two processes.

Optionally, as explained above, for emergency operation, for example if temperature-based adjustment or control of the heating is not possible during heating operation, pressure-dependent control or adjustment of the heating device can also be carried out during heating operation, in particular, for example, by reducing the heating output.

The present disclosure also provides a system for processing data, wherein the system is configured to perform the method according to the present disclosure, in particular as described above.

The present disclosure also provides a dialysis system comprising the system for processing data and further comprising a fluid system comprising a degassing section for degassing fluid, a sensor for determining the pressure in the degassing section, and a heating device for heating fluid contained in the dialysis system. The dialysis system may optionally comprise an inlet valve and/or a fluid container and/or a level sensor.

In particular, the heating device can be configured to heat fluid in a heating container of the dialysis system, in particular comprising a heating rod or a heating tube.

The pressure sensor or pressure sensors can be arranged in one or more areas of the degassing section.

The degassing section can comprise a degassing chamber, a pump and a throttle. The pressure can, for example, be measured in the direction of flow between the throttle and the pump. The degassing chamber can be arranged in the direction of flow between the pressure measurement area and the pump.

Monitoring the pressure may involve at least one pressure sensor being arranged in the degassing section and measuring the pressure, e.g. at regular intervals or continuously, and providing measured values. Monitoring can include, for example, regular or continuous recording of measured values from one or more pressure sensors.

The degassing chamber can be configured in such a way that it has many corners and edges that facilitate the separation of air. It can be a standard degassing chamber in this field.

The present disclosure also provides a computer program product comprising instructions which, when the program is executed by a computer, cause the computer to perform the method according to the present disclosure, in particular as described above.

The present disclosure also provides a computer-readable medium comprising instructions which, when the program is executed by a computer, cause the computer to perform the method according to the present disclosure, in particular as described above.

The features and advantages described in connection with the method also apply mutatis mutandis to the data processing system, the dialysis system, the computer program product and the computer-readable medium.

### BRIEF DESCRIPTION OF THE DRAWINGS

Further examples and embodiments are explained below with reference to the following Figures.

FIG. 1 shows a schematic representation of a method according to the present disclosure;

FIG. 2 shows a schematic representation of a system according to the present disclosure;

FIG. 3 shows a schematic representation of a system according to the present disclosure;

FIG. 4 shows a schematic representation of a pressure curve in a degassing section;

FIG. 5 shows a schematic representation of a curve of the standard deviation of the pressure in a degassing section; and

FIG. 6 a schematic representation of a method according to the present disclosure.

### DETAILED DESCRIPTION

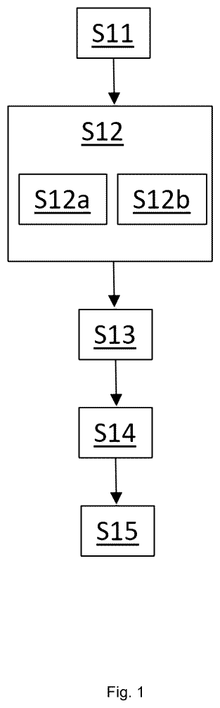

FIG. 1 schematically illustrates a computer-implemented method for a dialysis system for generating control data for pressure-based control of the dialysis system according to the present disclosure.

The method comprises, in step S11, monitoring the pressure in a degassing section of a fluid system of the dialysis system.

The method comprises, in step S12, generating control data when it is detected that the pressure meets at least one criterion. Generating the control data comprises generating 12a a heating device control signal S_{H} for a heating device of the dialysis system.

Optionally, generating the control data may further comprise generating S12b a valve control signal S_{V} for an inlet valve of the dialysis system which controls the fluid supply into the fluid system of the dialysis system, wherein the at least one criterion comprises an opening criterion, wherein, when the pressure fulfills the opening criterion, a signal for opening the inlet valve (opening signal) is generated, and/or wherein the at least one criterion comprises a closing criterion, wherein when the pressure fulfills the closing criterion, a signal for closing (closing signal) the inlet valve is generated.

The method may optionally comprise controlling S13 the heating device by means of the heating device control signal and/or controlling S14 the inlet valve by means of the valve control signal.

The optional control S13 of the heating device may comprise controlling the heating device in accordance with the heating device control signal with a predetermined delay and/or after at least one further criterion has been met and/or after user confirmation or immediately upon meeting the at least one criterion, in particular starting or stopping or adjusting the heating in accordance with the heating device control signal. The respective control signal can be such that the respective action specified by the control signal is executed with a delay, in particular with a delay specified by the control signal.

The optional control S14 of the inlet valve can comprise controlling the inlet valve in accordance with the valve control signal with a predetermined delay and/or after at least one further criterion has been met and/or after user confirmation or immediately upon meeting the at least one criterion, in particular opening or closing the valve in accordance with the valve control signal. The respective control signal can be such that the respective action specified by the control signal is executed with a delay, in particular with a delay specified by the control signal.

The method can optionally comprise a testing process S15 for testing a level sensor in a fluid container of the dialysis system, which can, for example, be located upstream of the degassing section in the direction of flow. In particular, the fluid container can be arranged so that fluid can flow directly from the fluid container into the degassing section. The monitored pressure and/or the control data can be used as the first input data and the measurement data of the level sensor as the second input data for the testing process.

For example, the testing process can output that the level sensor is functioning properly if both the measurement data of the level sensor and the pressure and/or the control data indicate that the amount of fluid in the dialysis system is below a predefined minimum amount, or if both the measurement data of the level sensor and the pressure and/or the control data indicate that a predefined minimum amount of fluid is present in the dialysis system. Otherwise, the testing process may indicate that the level sensor is not functioning properly.

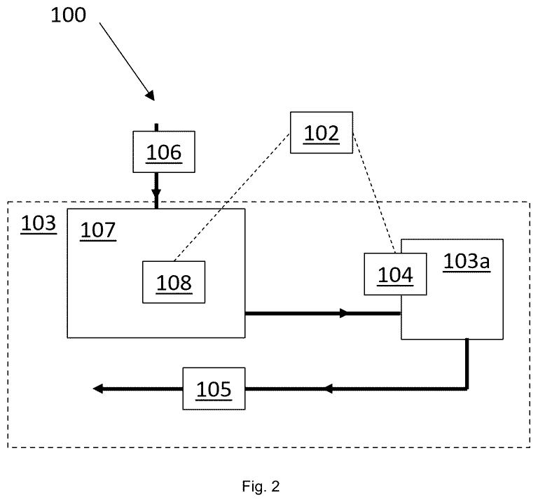

FIG. 2 schematically illustrates an example of a system 102 for data processing according to the present disclosure, wherein the system 102 is configured to perform the method according to the present disclosure, for example as described in connection with FIG. 1. The system may comprise one or more computing units on which the method is performed and/or controlled in a centralized or decentralized manner.

As shown in FIG. 2, the system 102 may be part of a dialysis system 100 according to the present disclosure, shown here schematically. The dialysis system may further comprise a fluid system 103 comprising a degassing section 103a for degassing fluid, a sensor 104 for determining the pressure in the degassing section 103a, and a heating device 105 for heating fluid contained in the dialysis system 100. Optionally, the system may comprise an inlet valve 106 and/or a fluid container 107 and/or a level sensor 108 in the fluid container 107, wherein the fluid container is part of the fluid system.

Further features and advantages of methods and systems according to the present disclosure are explained below.

The present disclosure discloses systems and methods for switching a heating process on and off, wherein the process is controlled/regulated as a function of a measured pressure.

The present disclosure describes a method for a dialysis system, i.e. a system for collecting, degassing and heating liquid as part of an extracorporeal blood treatment. In such a treatment, water can be continuously degassed and heated and then mixed with an alkaline and an acidic component to form the so-called dialysis liquid, which is then fed to a blood filter or dialyzer in order to enter into a diffusive exchange with blood.

The subject of the disclosure is, among other things, the provision of control signals for controlling components of a dialysis system, in particular the heating device and optionally a valve, on the basis of a pressure measurement, which can represent an indirect level measurement for a liquid container upstream of the degassing section in the direction of flow.

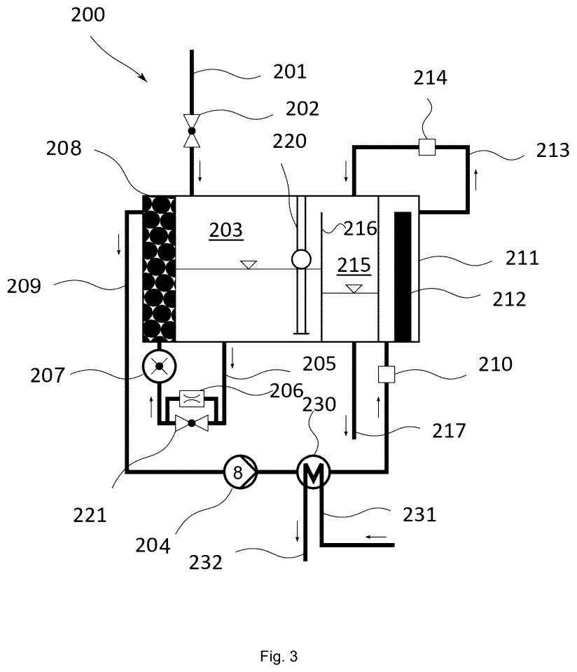

FIG. 3 schematically shows an exemplary system 200 (dialysis system) for collecting, degassing and heating liquid, in this case water for example. Water flows via the water inlet 201 into the flow chamber 203, also referred to as chamber 203, when the valve 202 is open. Driven by the pump 204, the water flows from the chamber 203 through the line 205 and the pressure measuring device 207 (also referred to below as sensor) into the degassing chamber 208. Upstream of the pressure measuring device 207 there is a throttle 206 to reduce the cross-section of the line 205. If necessary, the water can also be directed through the valve 221. Downstream of the pump 204, the water flows through a recuperator 230, in which it can absorb the heat of the incoming liquid in line 231. The flow chamber 203 can, for example, have an opening at the upper edge or in the upper cover of the flow chamber. Air can escape through this opening, which is expelled during degassing.

The heat-emitting liquid leaves the recuperator 230 through the line 232. The heated water then flows on through an optional temperature sensor 210 into a temperature control chamber 211, where it is further heated by a heating device 212. The water leaves the temperature control chamber 211 via line 213 and enters the withdrawal chamber 215, which is separated from the flow chamber 203 by a partition wall 216. A temperature measuring device 214 is located in or on line 213. If the fill level in chamber 215 exceeds the height of partition 216, the water can overflow into chamber 203. The tempered water can be withdrawn from the withdrawal chamber 215 through the line 217.

FIG. 3 also shows a line 222 which opens into the chamber 203 and is provided, for example, for circulation during a disinfection phase.

In the area of the line 213, a branch, not shown here, can optionally be provided through which water can be withdrawn. For example, a cartridge filled with sodium hydrogen carbonate and connected to the liquid system can be filled with water withdrawn in this way. The dissolved salt can then be used to produce the dialysis liquid. In addition to the line 217, there may also be a further withdrawal line.

The fill level in chamber 203 can be determined by the optional level sensor 220. If chamber 203 is detected as full, for example, valve 202 closes. It opens again as soon as chamber 203 is emptied or has been detected as empty or almost empty. This can be time-controlled or by observing other levels. A line 209 is also shown.

The present disclosure provides for the heating process to be carried out by the heating device 212 only when a negative pressure is measured at the pressure measuring device 207 while the pump 204 is running.

FIG. 4 shows an example of how the pressure measured by sensor 207 is approximately 0 mmHg or 0 kPa for a rotation rate of 1500 rpm of pump 204 when the flow chamber 203 is empty or almost empty. When the chamber 203 is full, the pressure stabilizes at a constant lower pressure value, which is dependent on the speed or delivery rate of the pump 204. Due to the residual volume in the chambers and lines, the pressure rises again with a time delay to a value of approx. 0 mmHg or 0 kPa when the chamber 203 has been emptied. The pressure drop (in this example at approx. 5 s) can be used to cause the heating device 212 to be switched on. The increase in pressure (in this example at approx. 80 s) can be used to cause the heating device 212 to be switched off.

For example, a threshold value can be used. If the chamber 203 is initially empty, i.e. the pressure is around 0 mmHg or 0 kPa, the heating process can be started as soon as the pressure falls below a value of −20 mmHg or −2.7 kPa, for example

If the chamber 203 is emptied, however, the heating process can be stopped as soon as the pressure exceeds the constant pressure value in the filled state (here approx. −320 mmHg or −42.7 kPa) by a certain value (e.g. 20 mmHg or 2.7 kPa).

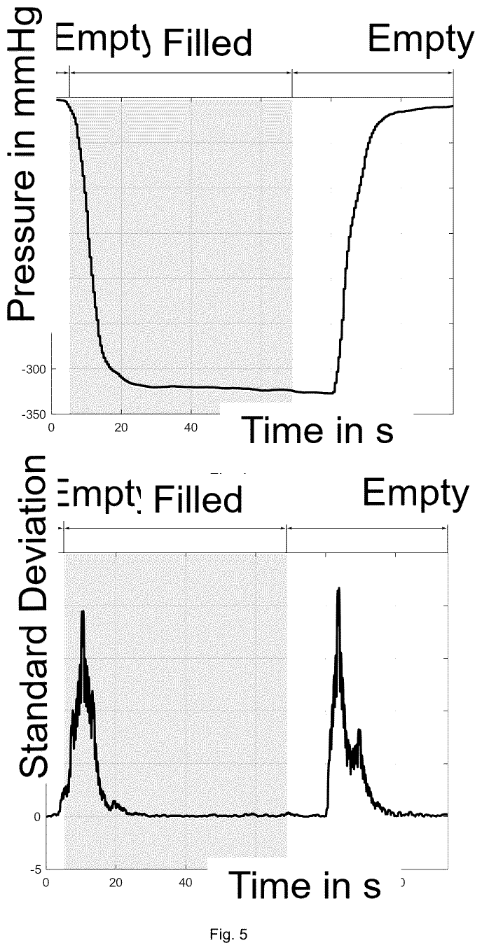

Alternatively, the moving standard deviation of the pressure can be evaluated. This is shown as an example in FIG. 5. Depending on which state was last present, i.e. whether heating was carried out or not, the heating process can be started or stopped when a threshold value of the standard deviation is exceeded (e.g. the value 5 for the specifications in mmHg). It is understood that the value depends on the selected pressure unit. If the pressure is specified in kPa, the threshold value of the standard deviation is of course correspondingly higher.

At least one of the lines 201, 205, 209, 213, 217, 231 and 232 may be configured as a hose or pipe or other connection.

Conventional level sensors are configured as floats that move along a rod in the tank with the fill level, for example. This can result in the float jamming and not being able to move any further. If the float remains in the upper position, for example, although the fill level is falling, this cannot be registered so that the inlet valve remains closed and the chambers run empty. If, on the other hand, the float remains in the lower position, for example, the chambers may overflow as the inlet valve remains permanently open.

By simultaneously observing the pressure at sensor 207, it is possible to detect if the float is jammed at level sensor 220.

If the float is jammed in the upper position, for example, the chamber 203 can run empty, which is noticeable by an increase in pressure at the measuring device 207 when the pump 204 is running.

A jamming in the lower position can also be detected if, for example, the temperature in the line 217 is also observed (measuring device not shown in FIG. 3). If the valve 202 is open, the water in chamber 203 flows after a certain time via the partition wall 216 into the withdrawal chamber 215. Since the water in chamber 203 is colder than the already heated water in chamber 215, a temperature drop in the line 217 would be detected or the temperature there is lower than at the measuring device 214.

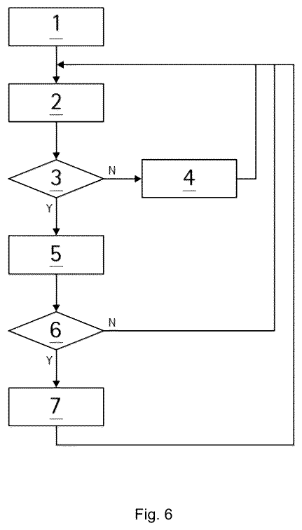

A further example of the present disclosure proposes the following simplified method steps for starting a heating process if no conventional level sensor is contained in a chamber of the feed tank, for example in a fluid tank immediately upstream of the degassing section. These steps are shown in FIG. 6 as a flow chart.

1. Starting pump 204 so that water (if present) can be drawn in from chamber 203. The bypass valve 221 is closed so that the water would have to flow through the throttle 206, which would lead to the build-up of negative pressure.

2. Measuring the pressure at the device 207.

3. Comparing the measured pressure with a threshold value. In the simplest case, this threshold value is 0 mmHg or 0 kPa and in particular between 0 mmHg or 0 kPa and −50 mmHg or −6.7 kPa. If the measured value is above the threshold value, the supply tank is empty (→go to step 4). If the measured value is below the threshold value, water has been pumped, which indicates that the supply tank is full (→go to step 5).

4. Opening the valve 202 in the event that the measured value was above the threshold value. Alternatively, opening can only take place for a predetermined time. After this time has elapsed, the valve 202 can close again automatically without having to wait for further conditions. Step 5 would be omitted in this case.

5. Closing valve 202 if it is not already closed (see step 4).

6. Optionally, before step 7, querying whether heating is desired by the program or by the user (the flow tank is always filled and the basic requirement for heating the water is met).

7. The heating process using heating device 212 is initiated and carried out.

Steps 2 to 7 can be repeated.

The following considerations can be used as examples for determining the predetermined time in point 4.

The setting of the pressure reducing valve at the water inlet is known to a certain extent. For example, the setting of the pressure reducing valve is often preset by the manufacturer with a specified tolerance. This makes it possible to estimate the amount of incoming water \dot{V}_{zu} when water inlet 202 is open (at constant line pressure), possibly within the specified tolerance. The inflow via the water inlet is between 30 and 40 ml/s, for example.

The volume ΔV of chamber 203 to be filled with the water inflow can be approx. 300 ml, for example (bottom of the chamber to the upper stop point of the float switch). With an inflow of 30 ml/s, the chamber is completely filled within 10 s. With an inflow \dot{V}_{zu} of 40 ml/s, the chamber is filled in 7.5 s. These values are calculated without the volume flow \dot{V}_{ab} flowing out through line 217.

A flow-dependent parameterization can be useful for the configuration of the opening duration of the water inlet valve 202.

The amount of outflowing water \dot{V}_{ab} at position 217 (from chamber 215 by means of a pump not shown) can be determined by flow balancing using balance chambers (also not shown). Flow balancing with balancing chambers can be performed using known methods. An overflow from chamber 215 into chamber 203 is possible via a bridge 216. The volume of overflowing water is generated by the volume flow \dot{V}_{EP} of the conveyor 204. For structural reasons, the volumetric flow \dot{V}_{EP} of the delivery device 204 is generally somewhat higher than the volumetric flow of the dialysis fluid pump (not shown) \dot{V}_{ab}. The water flowing out of chamber 215 reduces the volume passing over the bridge 216 through the delivery device 204 (pump) by the balanced flow.

The flow-dependent opening duration can therefore be determined dynamically as follows:

$$t = \frac{Δ V}{{\dot{V}}_{zu} - {\dot{V}}_{ab}}$$

The typical therapeutic dialysis fluid flow is between 300 ml/min (5 ml/s) and 800 ml/min (13.33 ml/s). According to the formula described, the greater the flow rate generated by the dialysis fluid pump, the greater the opening time t.

Furthermore, the dependency on the circulation valve must be considered (e.g. during disinfection). When flushing via the circulation circuit, the pumped volume Vab flows directly from the balance chambers or the flushing bridge back into chamber 203. Normally, no fluid flows into the drain so that the fill level does not drop. The volume flow \dot{V}ab thus becomes zero. The formula described remains valid. The inlet pipe for the circulation during the disinfection phase is marked with the reference sign 222 in FIG. 3. In the example shown in the figure, when the circulation valve is open, the fluid flows through line 222 back into chamber 203. Line 231, which leads to heat exchanger 230, and line 232, which is connected to the drain, are then no longer flowed through in this example.

Although the present disclosure is illustrated and described in detail in the drawings and the foregoing description, these illustrations and descriptions are to be considered exemplary and not limiting. The present disclosure is not limited to the disclosed embodiments. In view of the foregoing description and drawings, it will be apparent to those skilled in the art that various modifications can be made within the scope of the present disclosure.

1. A computer-implemented method for a dialysis system for generating control data for pressure-based control of the dialysis system, the computer-implemented method comprising the steps of:
monitoring a pressure in a degassing section of a fluid system of the dialysis system; and
generating control data when it is detected that the pressure meets at least one criterion,
wherein generating the control data comprises generating a heating device control signal for a heating device of the dialysis system.

2. The computer-implemented method according to claim 1, wherein:
the at least one criterion comprises a first criterion relating to a pressure value; and/or
the at least one criterion comprises a second criterion relating to a pressure change.

3. The computer-implemented method according to claim 2, wherein the at least one criterion comprises the second criterion, and the second criterion is a pressure increase and/or a pressure drop.

4. The computer-implemented method according to claim 1, wherein the at least one criterion requires an actual pressure value corresponding to a target pressure value.

5. The computer-implemented method according to claim 1, wherein the at least one criterion requires an actual pressure value falling below a predetermined pressure value or the actual pressure value rising above the predetermined pressure value.

6. The computer-implemented method according to claim 1, wherein the at least one criterion requires an actual pressure value changing by at least a predetermined amount.

7. The computer-implemented method according to claim 1, wherein the at least one criterion requires a moving standard deviation of the pressure rising above a predetermined value.

8. The computer-implemented method according to claim 1,
wherein the at least one criterion comprises a start criterion for starting the heating device, wherein when the pressure satisfies the start criterion, a start signal is generated as heating device control signal.

9. The computer-implemented method according to claim 8,
wherein the at least one criterion comprises the start criterion and the start criterion comprises that an actual pressure value falls below a predetermined pressure value or that an actual pressure value falls by at least a predetermined amount or that a moving standard deviation of the pressure rises above a predetermined value.

10. The computer-implemented method according to claim 1,
wherein the at least one criterion comprises a stop criterion for stopping the heating device, wherein when the pressure satisfies the stop criterion, a stop signal is generated as heating device control signal, and/or
wherein the at least one criterion comprises an adjustment criterion for adjusting operation of the heating device, wherein when the pressure satisfies the adjustment criterion, an adjustment signal is generated as heating device control signal.

11. The computer-implemented method according to claim 1, wherein at least one of:
the step of generating control data comprises generating a valve control signal for an inlet valve of the dialysis system which controls a supply of fluid into the fluid system of the dialysis system,
the at least one criterion comprises an opening criterion, wherein when the pressure meets the opening criterion, a signal to open the inlet valve is generated, or
the at least one criterion comprises a closing criterion, wherein if the pressure fulfills the closing criterion, a signal is generated to close the inlet valve.

12. The computer-implemented method according to claim 1 further comprising the step of controlling the heating device by the heating device control signal and/or controlling at least one inlet valve by at least one valve control signal.

13. The computer-implemented method according to claim 12,
wherein the step of controlling the heating device comprises controlling the heating device in accordance with the heating device control signal with a predetermined delay and/or after at least one further criterion has been met and/or after user confirmation or immediately upon meeting the at least one criterion, and
wherein the step of controlling of the at least one inlet valve comprises controlling the at least one inlet valve in accordance with a valve control signal with a predetermined delay and/or after at least one further criterion has been met and/or after user confirmation or immediately upon meeting the at least one criterion.

14. The computer-implemented method according to claim 1, further comprising a testing process for testing a level sensor in a fluid container of the dialysis system, wherein:
the pressure and/or the control data are used as first input data for the testing process,
measurement data from the level sensor is used as second input data for the testing process,
the testing process outputs that the level sensor is working properly when:
both the measurement data of the level sensor and the pressure and/or the control data indicate that an amount of fluid in the dialysis system has fallen below a specified minimum level, or
both the measurement data of the level sensor and the pressure and/or the control data indicate a presence of a specified minimum amount of fluid in the dialysis system,
and
the testing process otherwise indicates that the level sensor is not functioning properly.

15. The computer-implemented method according to claim 14, wherein control of heating and/or of an inlet valve takes place partially or completely according to the pressure.

16. The computer-implemented method according to claim 15,
wherein starting of heating is pressure-dependent according to the pressure and adjustment of heating during heating operation is independent of the pressure.

17. A system for data processing configured to perform the computer-implemented method according to claim 1.

18. A dialysis system comprising:
the system according to claim 17;
a fluid system comprising a degassing section for degassing fluid;
a sensor for determining the pressure in the degassing section; and
a heating device for heating fluid contained in the dialysis system.

19. A computer program product comprising instructions which, when the computer program product is executed by a computer, cause the computer to perform the computer-implemented method according to claim 1.

20. A computer-readable medium containing a computer program product comprising instructions, which, when the computer program product is executed by a computer, the computer executes the computer-implemented method according to claim 1.
